(19) United States
(12) Patent Application Publication
(10) Pub. No.: US 2013/0108699 A1
(43) Pub. Date: May 2, 2013

(54) HIGHLY DISPERSIBLE GRANULATE FOR THE PREPARATION OF FORMULATIONS OF HIGH DOSAGE ACTIVE SUBSTANCES AND PROCEDURE FOR OBTAINING HIGH DOSAGE ACTIVE SUBSTANCES THEREOF

(71) Applicants: Jose Antonio Matji Tuduri, Alcobendas (ES); Antonio Matji Arroquia, Alcobendas (ES); Luis Carvajal Martin, Alcobendas (ES)

(72) Inventors: Jose Antonio Matji Tuduri, Alcobendas (ES); Antonio Matji Arroquia, Alcobendas (ES); Luis Carvajal Martin, Alcobendas (ES)

(21) Appl. No.: 13/643,824

(22) PCT Filed: Apr. 26, 2010
(86) PCT No.: PCT/ES2010/070259

§ 371 Date: Jan. 8, 2013

(51) Int. Cl. A61K 9/20

(52) U.S. Cl. CPC A61K 9/2095
USPC 424/474; 514/570

(57) ABSTRACT

By mixing ibuprofen and a disaggregating agent, the invention involves using these components in a proportion ranging between 96% and 97% by weight for ibuprofen, and between 3% and 4% for the disaggregating agent, which may be Sodium croscarmellose, Crospovidone or Sodium carboxymethyl starch, as well as a mixture thereof. Following the mixing of the raw materials, the mixture is subjected to compaction, granulation and subsequent compression, in order to finally give the tablet a coating; this leads to a significant cost reduction and a significant reduction in the active principle release time, such that the pharmacological action is very fast in time.

### OBJECT OF THE INVENTION

The present invention relates to a new ibuprofen tablet that may be used as an anti-inflammatory agent, i.e. as a pharmacological product. Therefore, it is a product that falls within the pharmaceutical sector and, in particular, the physical-chemical processes for the galenic formulation of ibuprofen tablets.

The object of the invention is to obtain high-concentration tablets, with a high disaggregation rate of the active principle; specifically, with a maximum pharmacological response in the shortest possible time, all with a reduced manufacturing cost.

The invention also relates to the procedure for the manufacturing of said tablet.

### BACKGROUND OF THE INVENTION

The formula of ibuprofen is:

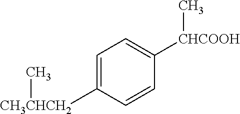

It is a fatty and acid product which in the market is presented as an impalpable powder.

Its melting point is between 73° C.-75° C., for which reason the powder may melt during the manipulation thereof or during the treatment of the tablet manufacturing processes (granulation, compaction and/or compression).

Due to its fatty character and low melting point, it is difficult to manufacture the tablets, and to release it once the tablets have been prepared.

On the other hand, when the product melts, the particle sizes of the re-solidified product are generally greater than the particle sizes of origin, which causes unmixing, adhesion to the walls and changes in the release process of the ibuprofen contained in the tablet.

In order to solve these problems, it is usually formulated by adding diluents and adsorbents to:
Be able to easily granulate or compact.
Prevent the tablets from adhering to the walls or surfaces that contain the granulate or the powder, due to melting, during the granulation steps (wet and/or dry granulation and compression).

A study of the ease of melting of ibuprofen due to heat reveals that it has a very low specific heat and a very low “latent heat of fusion”.

When ibuprofen is diluted with other components in a proportion of 20%-25% or greater and said mixture is granulated, the tendency to melt during the granulation and/or compression steps is diminished and/or disappears.
Make it possible for the ibuprofen tablet to release (in vitro) >85% in less than 15 min according to the USP specifications (Buffer pH 7.2 according to apparatus no. 1 [blades] at 150 rpm).

The rate of release of the ibuprofen in the tablet is of great importance, since, given that it is an anti-inflammatory agent (analgesic), it is desirable for it to obtain the greatest pharmacological response in the shortest possible time

In general, acids may be absorbed by passive absorption in the stomach and the first section of the intestine, for which reason the response of ibuprofen may be immediate, if it is released in a very short period of time.

Therefore, the current state of the art involves manufacturing a granulate by aqueous route or by the compaction of a mixture that has approximately 80% of active principle or less, and, subsequently, compressing that granulate.

The tablets thus obtained, with a maximum proportion of active principle of 70%-80%, are coated with different polymers, such as HPMC or similar.

Using these techniques, it is possible to obtain tablets that have an 85% release time equal to or less than 15 min, and, thus, make it possible for the pharmacological action to be very quick in time.

### DESCRIPTION OF THE INVENTION

The invention represents a technological advance in this field, since it improves the release time of ibuprofen, with the beneficial effects that this entails.

More specifically, the tablet proposed by the invention focuses its characteristics on the fact that it incorporates two components, which participate therein with the following proportions:
Ibuprofen between—96% and 97%
Disaggregating agent—between 4% and 3%

As a disaggregating agent, one or a mixture of the following products may be used:
Croscarmellose
Crospovidone
Sodium carboxymethyl starch (Primogel)

The procedure for obtaining and manufacturing the aforementioned tablet involves subjecting the mixture of the two aforementioned products to a dry compaction phase, preferably by means of a refrigerated roller compactor, in order to subsequently transform said compacted product into a granulated product, with the aid of a rotary or rolling granulator.

This granulated product may be directly compressed, without adding any other excipient, or it may be mixed with Aerosil 200 in a 99:1 proportion in order to be compressed.

The tablets thus obtained may be 200-, 400-, 600- or 800-milligram tablets, but the granulate obtained prior to the product compression phase may be directly packed in single-dose sachets.

The tablets obtained by means of the procedure of the invention, with any of the aforementioned dosages, may be coated with a protective film of an appropriate nature.

The tablets thus obtained have a rate of dissolution of less than 15 minutes for 85%, according to USP specification XX1V.

### PREFERRED EMBODIMENT OF THE INVENTION

Starting, as previously stated, from ibuprofen in a percentage ranging between 96% and 97%, and a disaggregating agent in a proportion ranging between 4% and 3%, using any of the aforementioned disaggregating agents, i.e. Croscarmellose, Crospovidone or Sodium carboxymethyl starch, following the mixing of said components, the mixture is compacted in a refrigerated roller compactor at a pressure ranging between 70 and 130 kN, to obtain a compacted wafer that is broken down with a rotary or rolling granulator equipped with a 1.5-to-2-mm sieve.

The refrigerated rollers must not exceed 25° C., such that the ibuprofen-disaggregating agent mixture does not heat or melt during the compaction action. The refrigeration of the rollers is a critical point in the production process.

The refrigeration may be performed with tower water at 10° C.-25° C. or with “Cheeler” water at 7° C.-10 ° C.

In summer, refrigeration with tower water presents temperature problems, exceeding the aforementioned limit of 25° C.; for this reason, the rollers are not appropriately refrigerated and this causes alterations in the nature of the granulate.

Refrigeration with “Cheeler” water may produce condensations of water in the rollers when the machine is off. In order to avoid this problem, the start-up of the machine entails the automatic opening of the refrigeration of the rollers and vice-versa.

In sum, the colder the rollers, the better the quality of the granulate obtained.

This is due to the fact that the activity of the mixing water increases as the temperature decreases, and the greater the Aw, i.e. the water activity, the greater the hardness of the compact due to the formation of hydrogen bridges with the residual water in the powder.

The residual water in the powder is primarily contributed by the disaggregating agent, and is capable of forming hydrogen bridges with the acid groups of ibuprofen, being greater the greater the water activity (Aw).

On the other hand, 90% of the granulometry of the starting ibuprofen must be less than 70 μm in diameter.

The selection of this ibuprofen with such small particle sizes in order to manufacture tablets is due to the fact that it is desirable to obtain a rapid rate of dissolution under the analytical assay conditions specified by the USP, since for smaller particle sizes the rate of dissolution of the tablet must be greater.

But this is only the case when the granulate in contact with the water is dispersed in particle sizes approximately equal to the original ones prior to the compaction. In order for this to be fulfilled, it is necessary that the mixture does not melt during the compaction process.

In order to verify this hypothesis, the following assay has been designed:

10 g of granulate are added in a 1% Sodium lauryl sulfate solution (100 ml) and it is stirred for 5 min.

After this time, the suspension is run through a sieve of between 70 and 100 μm.

The sieve is dried and the percentage of granulate that has been retained in the sieve is determined.

At least 90% of the powder must run through said sieve.

Once the test has been performed with 3 different batches, it is verified that between 91% and 93% of the product runs through a 100-μm sieve.

Therefore, it may be asserted that at least 91% of the granulate is dispersed in particles that are approximately equal to the starting ones.

As regards the granulometries of the granulate:

The ibuprofen-disaggregating agent mixture (97:3 or 96:4) is compacted with a refrigerated flat roller compactor, working with the following operating constants:

| | | |
|---|---|---|
| Feed rate: | ~50 | kg/hour |
| Roller pressure: | 70-120 | kN |
| Roller velocity: | 12-14 | rpm |
| Granulator (sieve): | 1.5 | mm |

The selected machine operates at constant pressure regardless of the feed rate.

Under these operating conditions, the following granulometries were obtained (2 batches):

| | Batch 1 | Batch 2 |
|---|---|---|
| Retains 840 μm | 27.64 | 29.0 |
| Retains 420 μm | 51.80 | 46.2 |
| Retains 250 μm | 7.30 | 14.1 |
| Retains 125 μm | 12.3 | 9.4 |
| Runs 125 μm | 1.4 | 1.1 |

It must be noted that the granulate barely has any fines. If the granulate obtained had a number of fines (for 125 μm) greater than 5%, it would be necessary to separate the fines by means of compaction recirculation thereof in order to obtain the desired granulates.

As regards compression of the granulate:
Since ibuprofen is greasy and fatty, it does not require lubrication in order to be compressed.
Since the granulate is dispersed in approximately the same particle sizes as the original, it does not require an external disaggregating agent.

Therefore, the granulate obtained in the preceding operation may be compressed without adding any other component.

However, if the rate of production is very high and the granulate is heated during the compression action, it tends to melt and stick to the punches.

4 different forms or ways to handle the compression of the granulate have been tested:

### 1st Form—Slow Rotary Machine:

The granulate obtained is compressed in a machine at a low speed of rotation (16-20 rpm) at a hardness of >6 Kp (normally at a hardness of 8 to 10 Kp) and a disaggregation time of 2-4 minutes.

No problems were observed after 8 hours of constant operation.

No adhesion of the powder to the punches was observed.

### 2nd Form—Fast Rotary Machine.

The granulate obtained is compressed in said machine at a rate that is 70% of the nominal rate.

The operating conditions were:

| | | |
|---|---|---|
| Compression rate: | 70,000 | comp./hour |
| Pre-compression pressure: | 4 | kN |
| Compression pressure: | 9-10 | kN |
| Depth of pre-compression punch: | 3 | mm |
| Depth of compression punch: | 2.32 | mm |

The tablets obtained under these operating conditions were:

| | |
|---|---|
| Disaggregation time: | 2-3 minutes |
| Hardness: | ≧6 Kp (7-9 Kp) |

When the machine had been operating for approximately ½ hour, the tablets began to stick due to the heat produced in the machine during the compression action.

3rd Form—Equal to the 2nd Form, but the granulate was at 10° C.:

When the granulate was at 10° C., the adhesion effect of the powder to the punches disappears and it is not observed during 1 hour of operation.

### 4th Form:

Equal to the 2nd form, but 1% of adsorbent is added to the granulate.

A quantity of 1% of Aerosil 200 (6 mg/tablet) was added to the granulate.

Adhesion of the powder to the punch disappears and it is not observed during 2 hours of operation.

The tablets obtained have the same characteristics as the previous ones and the operating conditions are identical to those of the 2nd form described.

The disaggregation time of the tablet is approximately 2 minutes and the hardness is 8 Kp.

As regards the rate of dissolution of the tablets obtained:

Since it is expected that the activity of an anti-inflammatory and analgesic agent of the ibuprofen type be as quick as possible, the operating conditions that produce tablets with a disaggregation time >3 minutes have been discarded.

The hardness of the tablet does not vary when the operating conditions are 9 kN in a fast machine, or 15 kN, and tablet hardnesses ranging between 8 and 11 Kp are obtained.

However, the disaggregation times of the tablets are very different. In the 1st case (9 kN), the disaggregation times are always less than 3 minutes, whereas in the 2nd case the disaggregation time is >6 minutes.

This specification makes it possible to quickly adjust the compression machine without having to wait for the dissolution rate assay, which would make the industrialisation thereof practically impossible.

A study of the non-standard behaviour of ibuprofen tablets with granulates containing 96% of active principle was also performed, and the following conclusions were reached:
Such different behaviours with relatively small changes in pressure are due to the fatty character of ibuprofen.
In order for the tablet to disaggregate, it must have micropores on the surface, such that the water may penetrate at a high speed and the disaggregating agent may break the tablet by swelling, soaked in water. These micropores are easy to observe on the lower side of the tablet with a magnifying glass.
When the micropores are reduced or cancelled due to the effect of the compression pressure, the disaggregation and subsequent release takes place by erosion, delaying and releasing the active principle according to a “controlled-release” behaviour of a lipid matrix. Said lipid matrix is the active principle itself.
An increase in the machine pressure does not lead to a greater hardness of the tablet, for which reason there is no direct relation greater hardness-greater disaggregation time.

The formulations described above entail a number of advantages, which primarily revolve around the following aspects:
Minimal primary cost, i.e. formulation cost.
Very fast process, which may be adapted to continuous processes.
Low cost of the process.
Low energy consumption.
Easy industrial implementation, with closed designs and very few space needs.
Short “cycle times”, with a response of less than 24 hours.

### EMBODIMENT OF THE INVENTION

For the manufacturing of tablets composed of 600 mg/tablet, the steps are the following:

### 1.Manufacturing of the Mixture

| Components | grams |
|---|---|
| Ibuprofen | 60,000 |
| Sodium croscarmellose | 2,400 |
| Total | 62,400 |

### Process

Sieve the ibuprofen and the Sodium croscarmellose through a 1-mm sieve and lift them to the 500-litre “Biconical” mixer.
Mix for 5 min.
Discharge the mixture and verify the uniformity of the mixture.

### 2.—Granulation by Compaction

Compact the mixture in a refrigerated roller compactor operating with the following constants:

| | | |
|---|---|---|
| Production | 45-50 | kg/hour |
| Roller speed: | 12-13 | rpm |
| Compaction pressure: | 70-100 | kN |
| Granulator sieve: | 1.5 | mm |

Verify that the fines <125 μm are less than 5%.

In the event that they are more than 5%, run the granulate through a continuous rotary sieve with a 200-250-μm mesh size.

### 3.—Compression

3.a. Low-Speed Machine

Compress at a speed of rotation of 14-18 rpm with the following constants:

| | | |
|---|---|---|
| Weight: | 600 | mg |
| Hardness | >6 | Kp |
| Disaggregation time | <3 | min. |

3.b. High-speed machine. Compress at a speed of rotation greater than 60 rpm and with a pre-compression station.
3.b.1—Mix the granulate with 1% of Aerosil 200 for 5 min.
3.b.2—Compress mixture 3.b.1 with the following constants:

| | | |
|---|---|---|
| Weight: | 630 | mg |
| Hardness | >6 | Kp |
| Disaggregation t. | <3 | min. |

The final machine pressure is 9 kN and the pre-compression pressure is 4 kN.

### 4.—Coating

Formula for 100,000 tablets containing 600 mg of ibuprofen

Suspension to be atomised:

| | | |
|---|---|---|
| HPMC of cps | 500 | g |
| Glycerin | 25 | g |
| Titanium oxide | 50 | g |
| Talc | 50 | g |
| Water sqf | 7,000 | g |

### 4.1 Preparation of the Suspension

### Process:

4.1.1.—Place approximately 6 litres of water in a tank under stirring (turbomixer) and, under constant stirring, add the 6-cps HPMC, stirring until the dissolution thereof.
4.1.2.—Add the glycerin, the titanium oxide and the talc.
4.1.3.—Make up to volume with water to 7,000 g.

### 4.2.—Atomisation of the Suspension

4.2.1.—Place the 625-mg tablets weight/tablet (100,000) in a perforated drum (Accela-cotta).
4.2.2.—Put the inlet air at 55° C.-60° C., keeping the drum in circulation at 1-2 rpm.
4.2.3.—When the tablets have a temperature of 38° C.-40° C., place the drum at 6 rpm and begin atomisation with the following operating constants:
Atomisation rate: 110-120 ml/minute
Inlet air temperature: 55° C.-60° C.
Outlet air temperature: 42° C.-46° C.
Atomisation pressure: 3-4 bar

Once the atomisation is complete, discharge the drum and collect the tablets in sachets, keeping them in trays.

The approximate atomisation time is between 1 hour and 1 h and 15 min per 63-63-kg load.

The increase in weight due to the coating must be between 5 and 6 mg/tablet.

1. Ibuprofen tablet, wherein the ibuprofen participates as the active component mixed with a disaggregating agent, characterised in that the ibuprofen participates in the mixture in a proportion ranging between 96% and 97%, whereas the disaggregating agent participates in a proportion ranging between 4% and 3%, by weight.

2. Ibuprofen tablet, according to claim 1, characterised in that the disaggregating agent is one or a mixture of the following components:
Sodium croscarmellose.
Crospovidone.
Sodium carboxymethyl starch (Primogel).

3. Procedure for obtaining the ibuprofen tablet of the preceding claims, characterised in that, following the mixing of said raw materials in the aforementioned proportions, said mixture is subjected to the following operational phases:
Compaction.
Granulation.
Compression.
Coating.

4. Procedure, according to claim 3, characterised in that the compaction phase is performed under dry conditions by means of a refrigerated roller compactor.

5. Procedure, according to claim 3, characterised in that the granulation phase is performed in a rotary or rolling granulator, with an appropriate sieve.

6. Procedure, according to claim 3, characterised in that the compression phase may be performed directly or from the granulated product, or said product may be mixed with Aerosil 200 in a 99:1 proportion.

7. Procedure, according to claim 3, characterised in that the granulated product is susceptible to being directly introduced in single-dose sachets.
